(19) United States
(12) Patent Application Publication
(10) Pub. No.: US 2010/0173885 A1
(43) Pub. Date: Jul. 8, 2010

(54) ORGANOMETEALLIC COMPOUNDS

(76) Inventors: Bruno Therrien, Neuchatel (CH); Govindasvamy Padavattan, Neuchatel (CH); Georg Sûss-Fink, Neuchatel (CH); Wee Han Ang, Cambridge, MA (US); Paul Joseph Dyson, Ecublens (CH); Lucienne Juillerat, Lausanne (CH); Frédéric Schmitt, Lausanne (CH)

Correspondence Address: KRIEG DEVAULT LLP, ONE INDIANA SQUARE, SUITE 2800, INDIANAPOLIS, IN, 46204-2079, US

(21) Appl. No.: 12/449,135

(22) PCT Filed: Jan. 25, 2008
(86) PCT No.: PCT/IB2008/050273

§ 371 Date: Jan. 20, 2010

(30) Foreign Application Priority Data
Jan. 26, 2007 (EP) 07101258.7

(51) Int. Cl. A61K 31/555; C07F 15/00; A61P 35/00; A61P 29/00; A61P 37/00

(52) U.S. Cl. 514/185; 546/4

(57) ABSTRACT

The present invention relates to novel organometallic compounds for use as a medicine, in particular in photodynamic therapy against various diseases, such as cancer or inflammatory and/or immune disorders. The compounds comprise a central porphyrin or phtalocyanine backbone to which ligand linkers coordinated to at least one transition metal are attached. Eta-5 or eta-6 arenes further bind to the transition metal. According to a preferred embodiment, the compound is a tetranuclear Ruthenium (+II) complex.

### TECHNICAL FIELD

The present invention relates to organometallic compounds, uses of the compounds in the treatment of cancer, in photodynamic therapy, as pharmaceutically active principles, to methods of treating cancer, inflammatory and/or immune disorders.

### BACKGROUND OF THE INVENTION AND PROBLEM TO BE SOLVED

Cisplatin, usually in combination with other drugs, is still the treatment of choice for more than 50% of all cancer patients. It shows good activity against testicular, ovarian, oropharyngeal, bronchogenic, cervical and bladder carcinomas, lymphoma, osteosarcoma, melanoma and neuroblastoma. However, it exhibits high toxicity and in general the treatment generates undesirable side-effects, such as nephrotoxicity and gastrointestinal toxicity. Moreover, it is inactive against many cancer cell lines and also against metastatic cancers. Therefore, the development of new drugs, such as metal-based ones, with reduced side-effects, and with equivalent or better activity than cisplatin has been the focus of much research, and is also an objective of the present invention.

In photodynamic therapy (PDT) of cancer, porphyrin derivatives are used as photosensitising agents. By exploiting photochemical and photobiological processes initiated by the porphyrin core, irreversible damages are created to tumor cells. In U.S. Pat. No. 7,087,214 platinum(II)-porphyrin complexes are disclosed, in which chemotherapeutic and photodynamic properties are combined. Of course, the above-indicated disadvantages are also observed in the complexes of this reference.

Many other compounds have been suggested in different types of therapies against different types of cancers. For example, Jang-Qiang Wang et al. “Porphyrin-carborane organometallic assemblies based on 1,2-dicarba-closo-dodecacarborane (12) ligands”, Chem. Comm., 2006, 162-164, propose rhodium and zinc containing complexes in Boron Neutron Capture Therapy. In WO 02/40494, a completely different approach is disclosed, based on ruthenium-aryl-compounds comprising a 1,3,5-triaza-7-phosphadamatane ligand, which are used in cancer therapy. So far, however, compounds resulting from these recent efforts have not yet reached the stage of marketed products.

In view of the prior art, it is an objective of the present invention to provide other, different compounds that can be used in the treatment of cancer, for example in photodynamic therapy. Preferably, these compounds have a reduced general toxicity if compared to cisplatin. It is desired that these compounds exhibit less side-effects than cisplatin and are also effective against primary cancers and metastases.

It is a further objective of the present invention to provide a means for treating inflammatory and/or immune disorders.

### SUMMARY OF THE INVENTION

In a first aspect, the present invention provides an organometallic compound comprising a structure of formula (I):

[MₙMc(A)ₙ(L)(B)ₚ(C)_{q}] (I)

which is charged or neutral and which may be present in the form of a salt and/or an optically resolved enantiomer, and,
wherein:
M is a transition metal selected from Ru, Rh, Os, Ir, and Fe;
n is an integer of 1 to 12 if L corresponds to formula (II) below and an integer of 1 to 16 if L corresponds to formula (III) below and defines the number of metal atoms M present in compound (I);
Mc is an optional metal in the centre of L, which may be selected from the same metals as M, and from metals including Zn, Ni, Co, Cu, Ag, Au, Cd, Hg, Pd, and Pt.
A and L are ligands of M;
B and C are, independently of each other, optional ligands of M, with p and q, independently of each other, being 0 or an integer of 1 to n.
wherein, if n>1, all n ligands A and all n metals M in the compound of formula (I) can be the same or different; and, accordingly, wherein, if p and/or q>1, all p ligands B and/or all q ligands B, respectively, in the compound of formula (I) can be the same or different;
A is an η⁶-arene or a η⁵-cyclopentadienyl, or a η⁵-cyclopentadienyl derivative;
B and C are, independently of each other, freely selectable ligands capable of binding to M,
wherein:
B and C may be covalently connected with each other so as to form a single, bidentate chelating ligand (B-C), or, alternatively, any one or both of B and C, may be covalently bound to A, so as to form a multidentate chelating ligand (A-C), (A-B) or (A-B-C);
   L is a porphyrin derivative of formula (II) or a phtalocyanine derivative of formula (III) below:

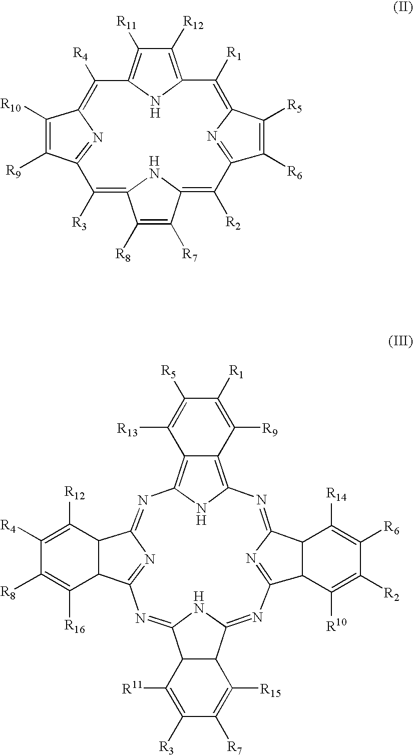

wherein:
m residues of R₁-R₁₂ if L=(II) or of R₁-R₁₆ if L=(III) are linkers comprising, all m residues together, at least n donor groups for M, wherein each of said at least n donor groups is selected independently from S-, O-, N- and/or P-donor groups, and,
m is an integer of 1 to 12 if L=(II) and an integer of 1 to 16 if L=(III),
   wherein:
      two or more (r) of said m residues of R₁-R₁₂ if L=(II) or of R₁-R₁₆ if L=(III) may, independently of the remaining (m-r) residues, be covalently connected with each other to form a system comprising at least one ring fused to the porphyrin structure of formula (II) or to the phtalocyanine structure of formula (III), respectively;
and, wherein:
(12-m) residues of R₁-R₁₂ if L=(II) or (16-m) of R₁-R₁₆ if L=(III), respectively, are selected, independently of each other, from H, C1-C20 alkyls, C2-20 alkenyls, C2-C20 alkynyl, C6-C22 aryls, said alkyls, alkenyls, alkynyls and aryls being optionally substituted and optionally comprising one or more heteroatoms, said optionally substituted alkyls, alkenyls, alkynyls being linear or cyclic.

In a second aspect, the present invention provides the use of the compounds of the present invention as pharmaceutically active principles, in particular as medicaments.

In a third aspect, the present invention provides a method of treatment of cancer, the method comprising the step of administering to an individual a pharmaceutically active quantity of the compound of the present invention.

The compounds according to the present invention were found to have excellent properties in photodynamic therapy.

### BRIEF DESCRIPTION OF THE DRAWINGS

FIG. 1 shows UV-visible absorption spectra of TPP (5,10,15,20-tetra(4-pyridyl)porphyrin) and compounds 1-7 of the present invention in dichloromethane at 298 K.

FIG. 2 shows the structures of the ruthenium compounds 1-5 according to the present invention and schematically indicates their synthesis.

FIG. 3 shows the structures of the osmium, iridium and rhodium analogs (compounds 6-8) according to the present invention.

FIG. 4 shows survival dose-response curves in the dark of a melanoma cell lines (ME300), after 24 h of exposure to various concentrations of organometallic porphyrin complexes 1-7 according to the present invention.

FIG. 5 shows photodynamic sensitivities for compounds 1-7 in Me300 melanoma cells. Survival by MTT test was assessed for cells exposed to increasing doses of light at 652 nm wavelength (0 J/cm² in white, 5 J/cm² in light gray, 15 J/cm² in dark gray and 30 J/cm² in black). Cells were incubated with photosensitizers (10 μM) for 24 h before light treatment.

FIG. 6 shows photoxicity 24 h after irradiation at 652 nm in RAW macrophages loaded with the compounds of the present invention. The efficacy of the compounds of the invention against inflammatory cells shows the use of the compounds in the treatment of inflammatory and/or immune disorders.

### DETAILED DESCRIPTION OF THE PREFERRED EMBODIMENTS

The compounds of the present invention comprise a structure of formula (I), [MₙMc(A)ₙ(L)(B)ₚ(C)_{q}], wherein M is selected from Ru, Rh, Os, Ir, and Fe, including combinations of two or more of these. For example, if n is 4, M₁ and M₃ may be Ru, with M₂ and M₃ being Os in the compound of the invention. Preferably, M is selected from the group consisting of Ru, Rh, Os, Ir, and combinations including two or more of these. More preferably, M is selected from the group of Ru and Os and combinations thereof. While different metals M may be combined if n>1, the compound of the present invention preferably comprises only one transition metal M. Most preferably M is Ru. The transition metal M may be present at any oxidation state, for example +IV, +III, +II. Different oxidation states may be present within the same molecule. Preferably, M is at the oxidation state +II.

Preferably, the optional metal Mc is absent, the compound of formula (I) of the present invention corresponding to formula [Mₙ(A)ₙ(L)(B)ₚ(C)_{q}]. In other words, the compounds of the present invention are preferably free of a central metal Mc. However, it is easy to add a central metal to the compounds of the present invention, for example for the purpose of modifying the charge of the overall compound or for other reasons, including non-technical ones. Therefore, the present invention also encompasses compounds in which a central metal atom is present. The central metal Mc, if present, is coordinated to the centre of L, which is described in further detail below.

The letter n designates the number of metals M in the compound of the present invention, and is therefore an integer. Theoretically, n is only limited by the possibility of adding suitable ligand linkers to the central structure L (see below) and could therefore be in the ranges of 10-20, 3-30, 15-50 or even higher. However, simpler specimen of the present invention will generally comprise less metal atoms, n thus being in the range of 1-16 or 1-12. Preferably, n is an integer of 1-10, more preferably 2-8, even more preferably 3-6. Most preferably, n is 4.

L is a compound according to the structure of formula (II) or (III) shown above, wherein at least one of the residues R₁-R₁₂ in case of structure (II) and R₁-R₁₆ in case of structure (III) comprises at least one donor group, which binds, by a typical ligand bond, to the metal M.

For the purpose of the present invention, the reference to a group of consecutively numerated residues R_{X}-R_{Y}, for example in the phrase “at least one of the residues R₁₋₁₆” or “any of R₁₋₁₆” is understood as a reference to all residues included in the range. For example, “at least one of residues R₁-R₁₆” means at least one of the group of R₁, R₂, R₃, R₄, R₅, R₆, R₇, R₈, R₉, R₁₀, R₁₁, R₁₂, R₁₃, R₁₄, R₁₅, R₁₆”. “Two of R₁-R₁₆” means two different residues picked randomly from this group, and so forth.

With respect to L, the expression “a residue R” is to be understood as “one of the residues R₁-R₁₂ if L=(II) and one of R₁-R₁₆ if L=(111)”. The expression “any R” means “any R of R₁-R₁₂ if L=(II) and at least one of R₁-R₁₆ if L=(III)”, and so forth.

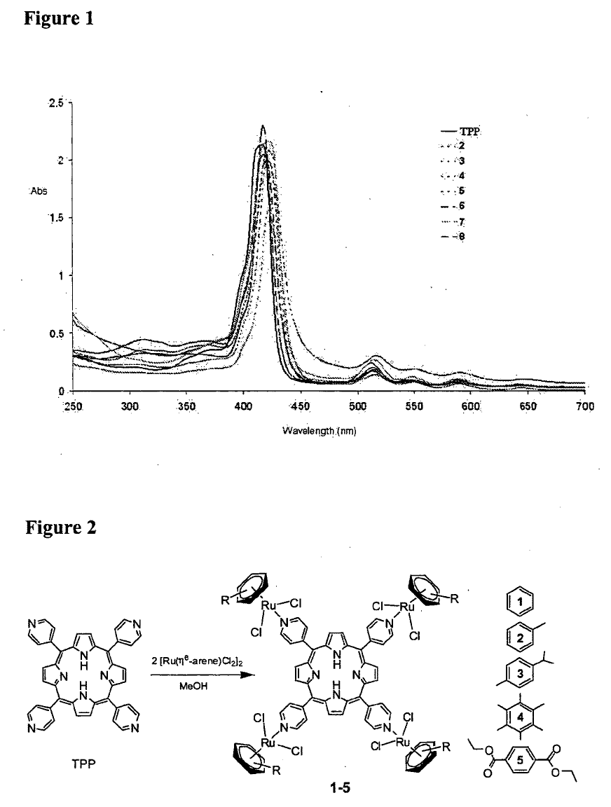

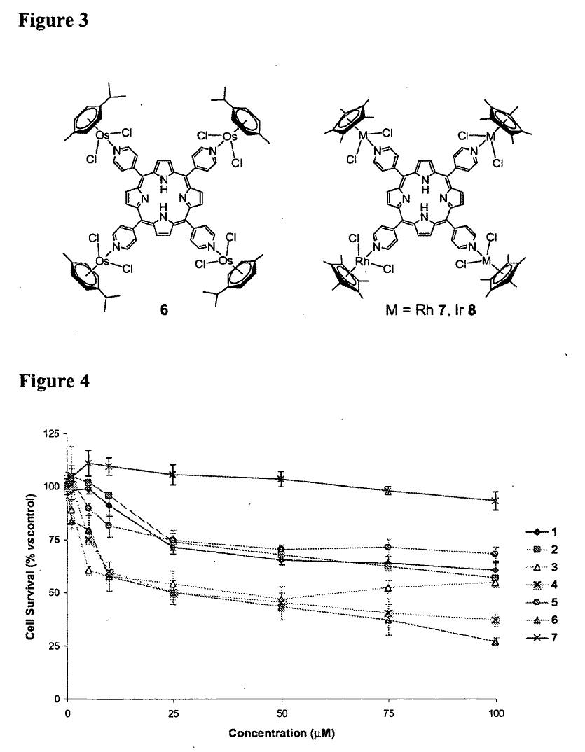

L may have one or more residues, namely m residues, carrying, altogether, at least n donor groups so that L functions as a ligand to all n metals M. In other words, any residue R, for example R₁, may carry one or more donor groups and thus form a ligand to an according number of metals M. It is also possible that any R comprises one or more chelating groups, so that two or more donor groups of the respective residue bind to the metal M, similar to the situation shown in US 2004/0023942, FIG. 1. Furthermore, one can envisage two residues R forming a chelating ligand for one M, similar to the situation in US 2004/0023942, FIG. 2. This document is entirely incorporated herein by reference.

As the skilled person will understand, at least one of the residues R of L, but potentially all of them, is a linker that comprises a donor group so as to bind to metal M. This linker is also referred to herein as “ligand linker” or “linker residue”. Now, the present invention does not intend to impose any limitation as to the structure of this linker. To date, a great number of such linkers is available and the mentioning of all of them would go beyond the information that can be listed in this document. However, for the purpose of providing a guideline, further indications concerning the possible structure of the ligand linker are given below. It is recalled, however, that there are nearly no limits with respect to the concrete structure that is used, and which one(s) of the residues R of L forms a ligand linker.

Accordingly, any such linker residue R of L is preferably a C1-C22, more preferably C2-C15 and most preferably C3-C10, linear, branched and/or cyclic hydrocarbon comprising an S-, O-, N-, and/or P-donor group. Of course, the linker residue may be further substituted and may comprise one or more heteroatoms.

S-donor groups are groups which bind to a metal M via a sulphur atom. They are well known in the art and include: sulfoxide groups (R^{S1}R^{S2}SO), thioether groups (R^{S1}R^{S2}S), thiolate groups (R^{S1}S⁻); sulfinate groups (R^{S1}R^{S2}SO); and sulfenate groups (R^{S1}SO⁻), wherein R^{S1} and R^{S2} are independently selected from C1-C20 alkyl, C2-C20 alkenyl, C2-C20 alkynyl and C5-C22 aryl, which may be linear, cyclic and/or branched, which are optionally substituted and which optionally comprises one or more heteroatoms, and where at least one of R^{S1} and R^{S2} is a bivalent substituent bound to L, thus forming a corresponding residue R. Of course, it is possible that if two residues R^{S1} and R^{S2} if both present are present, are formed from two residues R of L.

Residues comprising O-donor groups are residues which bind to a metal M via an oxygen atom. They are well known in the art and include: carboxylate ligands (R^{C}CO₂⁻); and sulfonate ligands (R^{S1}SO₃⁻), wherein R^{C} and R^{S1} are defined as R^{S1} and R^{S2} above.

Residues comprising N-donor groups are residues which bind to a metal M via an nitrogen atom. They are well known in the art and include nitrile ligands (N≡C—RN); azo ligands (N═N—RN), aromatic N-donor ligands, and amine ligands (NR^{N1}R^{N2}R^{N3}), for example.

In both nitrile and azo ligands R is defined as R^{N1}-R^{N3}.

Aromatic N-donor ligands include optionally substituted pyridine, pyradizine, pyrimidine, pyrazine, imidazole, purine, 1,8-naphthydrin, chinoxaline, chinazoline, pteridine, to mention a few examples.

With the amine ligands, R^{N1}, R^{N2} and R^{N3} may be independently selected from H and C1-C10 alkyl, C2-C20 alkenyl, C2-C20 alkynyl and C5-C22 aryl, they may be linear, cyclic and/or branched, and optionally be substituted and optionally comprising one or more heteroatoms, with the proviso that at least one of R^{N1}, R^{N2} and R^{N3} is a bivalent substituent bound to L, thus forming a corresponding residue R. It is, of course, possible that two or all three of R^{N1}-R^{N3} are connected to each other to form a cyclic residue comprising an N-donor group.

According to an embodiment of the present invention, each of said linkers comprises, independently of the others, at least one aromatic N-ring or at least one amine functioning as said N-donor group.

Residues comprising P-donor groups are residues which bind to a metal M via an phosphorous atom. They are well known in the art and include phosphine ligands (R^{P1}R^{P2}R^{P3}P) and phosphite ligands P(OR)₃, wherein the 3 residues (OR) of the phosphite ligand may be different, resulting in a formula (P(OR^{P1})(OR^{P2})(OR^{P3})), wherein R^{P1}, R^{P2}, and R^{P3} in said phosphine or phosphite ligand are, independently of each other selected from C1-C20 alkyl, C2-C20 alkenyl, C2-C10 alkynyl and C5-C22 aryl, they may be linear, cyclic and/or branched, and optionally are substituted and optionally comprise one or more heteroatoms, with the proviso that at least one of R^{P1}, R_{P2} and R^{P3} is a bivalent substituent bound to L, thus forming a corresponding residue R. It is, of course, possible that two or all three of R^{P1}-R^{P3} are connected to each other to form a cyclic residue comprising an P-donor group. An example of such a compound is PTA (1,3,5-triaza-7-phosphaadamantane).

A particularly preferred linker ligand R of L comprising an N-donor group is the ligand of formula (VI).

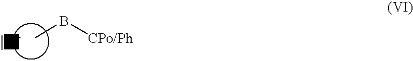

wherein:
the circle represents a mono- or polycyclic system, optionally substituted, comprising at least one N-heteroatom, indicated as N, which provides an electron pair, indicated as vertical line, enabling attachment to M;
B is an optional connecting unit, which may be selected from alkylene, alkenylene, alkynylene and arylene, which is optionally substituted, which optionally comprises one or more heteroatoms and has 0-15, preferably 1-8 carbon atoms;

The dotted line represents a bond to a carbon of the porphyrin or phtalocyanine of formula (II) or (III), indicated as CPo/Ph, which is itself not part of the linker, but a carbon carrying any residue R of L.

Preferably, in formula (VI), the circle is a heterocyclic ring, for example a heterocyclic arene.

Preferably, it is a 5- or 6-membered heterocyclic ring, for example arene. Preferably, B is a C₁-C₁₆, more preferably a C₂-C₄ alkylene.

According to a particularly preferred embodiment, any one of the ligands R of L is a N-donor ligand comprising the structure of formula (VII) below:

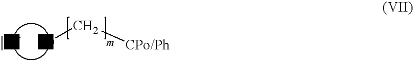

the circle represents a mono- or polycyclic system, which is optionally substituted, comprising at least one N-heteroatom, indicated as N, which provides an electron pair, indicated as vertical line, enabling attachment to M;
D is a carbon or nitrogen atom;

The dotted line represents a bond to a carbon of the porphyrin or phtalocyanine of formula (II) or (III), indicated as CPo/Ph, which is itself not part of the linker, but a carbon carrying any residue R of L.

m is 0-10, preferably 1-6, most preferably 3-4.

The cyclic system may, for example, be selected from pyridine, imidazole, purine, pyrazine, pyrimidine, 1,8-naphthydrin, chinoxaline, chinazoline, pteridine. Preferably, the cyclic system is imidazole, resulting in N-donor ligands comprising structures as illustrated in formula (VIII) and (IX) below.

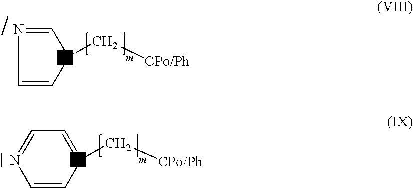

wherein m, the dashed line and CPo/Ph have the same meanings as for formula (VII) above.

According to another, preferred embodiment, one or more R of L is a linker ligand of formula (X), more preferably (XI) below.

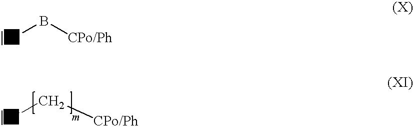

wherein Y can be a N-, O-, S- or P-donor group such as defined above, for example —NH₂ or —COO⁻; B, m, the dashed line, and CPo/Ph have the same meanings as for formula (VI) or (VII) above.

Two or more residues R of L may bind to the same metal M in compound (I), acting as a bidentate chelating ligand. For example, two ligands may have a structure of formula formulae (XII) or (XIII) below.

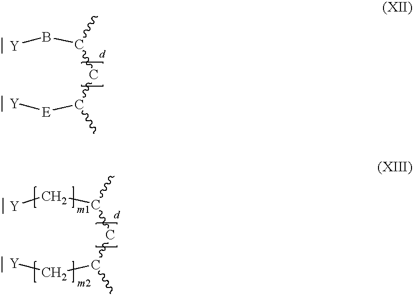

wherein Y is as in (X) above; B and the dashed line are as in formula (VI) above; E is selected from alkylene, alkenylene, alkynylene and arylene, which is optionally substituted, which optionally comprises one or more heteroatoms and has 1-15, preferably 2-8 carbon atoms, wherein B and E may be the same or different; C denotes carbons of the porphyrin or phtalocyanine backbone of formulae (II) or (III) to which the linker ligand is covalently bound (dashed line), and the curved lines design bonds between carbons of said (II) or (III) backbone, which may be single or double bonds, but preferably comprise conjugated double bonds. Small m1 and m2 may are integers of 1-10, preferably 2-5, and may be the same or different. As is indicated by d, which is 0 or an integer of 1-10, preferably 1-3, the residues carrying the donor group may be attached to neighbouring carbons C or may be separated by up to d carbons of the porphyrin or phtalocyanine backbone (II) or (III), respectively.

For providing another example, any residue R of L as shown in (II) or (III) can also be selected from residues having the structure of formula (XIV):

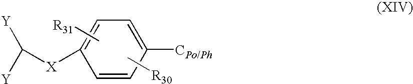

wherein the dotted line and CPo/Ph are defined as in formula (VI) above; Y is defined as in formula (X) above; R₃₀ and R₃₁ may independently of each other be selected from H, alkyls and alkenyls, for example; X may be selected from —[CH₂]ₘ— as defined in formula (VII) above, —O—, —S—, —NH—, and from —N(-alkyl)-. Preferably, Y is —COO— or NH₂.

As the examples above illustrate, the constitution of the linker ligands forming at least one of residues R₁-R₁₂ and R₁-R₁₆, respectively, is not restricted to a specific structure, but may be chosen by the skilled person's preferences.

The compounds of the present invention comprise an arene or cyclopentadienyl, or a derivative thereof, which is preferably bound by a η6- or η5-, respectively, bond to M. Preferably, the arene and/or the cyclopentadienyl derivative is a C6-C30, more preferably C6-C22, even more preferably C6-C12 arene or cyclopentadienyl derivative, optionally comprising one or more heteroatoms.

For example, A may be a mono-, oligo- or polycyclic system, comprising two or more rings fused together. Examples are of A thus include benzene, naphthalene, anthracene, indene, biphenylene, which are optionally substituted and which optionally comprise one or more heteroatoms.

According to a preferred embodiment of the organometallic compound of the present invention, is a compound of formula (IV) or (V):

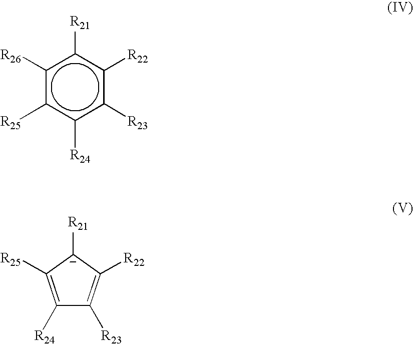

wherein, if A is (IV), any one of R₂₁-R₂₆, or, if A is (V), respectively, any one of R₂₁-R₂₅, is independently selected from H, C1-C20 alkyl, C2-20 alkenyl, C2-C20 alkynyl, C6-C22 aryl, said alkyl, alkenyl, alkynyl being linear or cyclic, and said alkyl, alkenyl, alkynyl and aryls being optionally substituted and optionally comprising one or more heteroatoms, wherein any of the residues R₂₁-R₂₆ or R₂₁-R₂₅, respectively may be covalently connected with one or more other residues of R₂₁-R₂₆ or R₂₁-R₂₅, respectively, to form one or several rings fused to the ring of formula (IV) or (V), respectively.

Preferably, if A is (IV) any one of R₂₁-R₂₆, or, if A is (V), respectively, any one of R₂₁-R₂₆, is selected, independently from each other, from H, C1-10-alkyl, C1-10-alkoxy, C1-10 ether, and C1-10 ester.

For example, A is benzene, 1,2-dimethylbenzene, 1,3-dimethylbenzene, 1,4-dimethylbenzene, 1,3,5-trimethylbenzene, 1,2,4-trimethylbenzene, durene, methyl-benzene, p-cymene, hexamethylbenzene, cyclopenta-1,3-diene, 1,2,3,4,5-pentamethylcyclopenta-1,3-diene, diethylterephtalate.

The above definitions also encompass the possibility that one or more of substituents R₂₁-R₂₆ may be provided by a bioactive compound, covalently bound to the benzene or cyclopendtadienyl structure of (IV) and (V). Accordingly, the substituent R₂₁-R₂₆ may be itself be a medicament, for example a medicament useful in the treatment of cancer. WO2007/128158, which is expressly and entirely incorporated herein by reference, disclose arenes, which have inhibitors of resistance pathways, related compounds and/or derivatives bound to an aryl, such as a benzene or cyclopentadienyl ring.

B and C are, as indicated above with respect to the structure of formula (I), [MₙMc(A)ₙ(L)(B)ₚ(C)_{q}], independently of each other, optional ligands of M, with p and q, independently of each other, being 0 or an integer of 1-n.

B and C are uncommitted ligands capable of binding to M, which means that they may be freely selected. For example, they may be provided by solvent molecules. According to a preferred embodiment, A and B are, independently of each other, selected from H, F, Cl, Br, I, OH, OMe, OEt, OBu, OH₂, CH₃CN, (CH₃)₂CO, DMSO, THF, CO, CF₃SO₃, C₂O₄, phosphine ligands as generally defined above with respect to ligands comprising an P-donor group, for example PMe₃, PEt₃, PBu₃, PPh₃, PCy₃, acetate, acetylacetonate, pyridine, pyrimidine, pyrazine, imidazole, pyridazine, phthlalazine, quinoxaline, 1,5-naphthyridine, 1,8-naphthyridine, 2,2′-bipyrimidine, phenazine, azobenzene, 2,2′-azopyridine, 2,3-di-(2′-pyridino)quinoxaline, diphenylphosphineethane, pyrazinecarboxylic acid, pyrazinedicarboxylic acid, DABCO, ethylenediamine, 1,3,5-hiaza-7-phosphatricyclo[3.3.1.1^{3,7}]decane (PTA).

More preferably, B and C are, independently of each other, selected from H₂O, Cl, Br, I, CO, SH, OH, Pyridine, PPh₃, PTA.

The compound (I) of the present invention may comprise p ligands B and q ligands C. All q ligands B of a compound (I) may be the same or different, and all p ligands C of said compound (I) may be the same or different. Preferably, all p ligands B are identical. Similarly, all q ligands C are preferably identical.

Small p and q may be different, but preferably p=q. Even more preferably, n=p=q, which means that for every metal M, one B and one C is present, binding to that metal. B and C may be covalently connected with each other so as to form a single, bidentate chelating ligand (B-C). Examples of such chelating ligands are ethane-1,2-diamine, propane-1,2-diamine, benzene-1,2-diamine, cyclohexane-1,2-diamine, malonate, 2,2-bipyridine, pyrazinecarboxylic acid, oxalate, quadratic acid, quinoxaline, and 2,2-bipyrimidine.

Alternatively, B and C, may be covalently bound to A, so as to form a multidentate chelating ligand (A-B), (A-C), or (A-B-C). (A-C) and (A-B) include compounds of formula (IV) and (V), in which one of the residues R₂₁₋₂₆ and R₂₁₋₂₅, respectively, comprises an S-, O-, N- and/or P-donor group as defined above. To provide some arbitrary examples of ligands (A-B) or (A-C) one could mention 2-phenylethanamine, 2,2-bipyridine, 2,2-bipyrimidine.

(A-B-C) include compounds of formula (IV) and (V), in which two of the residues R₂₁₋₂₆ and R₂₁₋₂₅, respectively, comprise an S-, O-, N- and/or P-donor group as defined above. An example for such a compounds would be 2,2′-(1,4-phenylene)diethanamine, 2,2-bipyridine, 2,2-bipyrimidine.

According to preferred embodiment of the present invention B and C are separate compounds individually bound to M. Preferably, B and C are the same compound.

In the present specification reference to “substituents” is repeatedly made, for example in the expression “optionally being substituted”, in particular with respect to residues, structures or compounds in particular residues R₁-R₁₂ of the structure of formula (II), residues R₁-R¹⁶ of the structure of formula (III), residues R₂₁-R₂₆ of (IV), R₂₁-R₂₅ of (V), residues of ligand linkers comprising S-, O-, N-, and/or P-donor groups, namely of R^{S1}, R^{S}, R^{C}, R^{N1}, R^{N2}, R^{N3}, with respect to ligand linkers (VI), (VII), (VIII), (IX), (X), (XI), (XII), (XIII) and (XIV).

These substituents may be selected, independently from each other within each residue, from linear, branched and cyclic C1-C20 alkyls, alkenyls, and/or alkynyls; halo (—Cl, —Br, —F, —I); keto (═O); hydroxy (—OH); carboxy (—COOH); amino (e.g. —NH₂); cyano (—C≡N); nitro (—NO₂); (mercapto (—SH); thiooxo (═S); sulfo (—SO₃H); sulfino (—SO₂H); sulfeno (—SOH). Substituents may comprise heteroatoms and may be further substituted as defined herein.

Heteroatoms, for the purpose of the present specification, include all heteroatoms, but in particular B, Al, Si, O, S, Se, N, P, As, halo, Li, Na, K, Be, Mg, Ca, Sr, for example. Preferred heteroatoms are O, S, N, P, and halo.

Examples of C1-C10 alkyls include saturated alkyls, such as methyl (C1), ethyl (C2), propyl (C3), butyl (C4), pentyl (C5), hexyl (C6), heptyl (C7), octyl (C8), nonyl (C9), decyl (C10); saturated linear alkyls such as methyl (C1), ethyl (C2), n-propyl (C3), n-butyl (C4), n-pentyl (C5), n-hexyl (C6), n-heptyl (C7), n-octyl (C8), n-nonyl (C9), n-decyl (C10), cyclic alkyls such as cyclopropyl propyl (C3), cyclobutyl (C4), cyclopentyl (C5), cyclohexyl (C6), cycloheptyl (C7), cyclooctyl (C8), cyclononyl (C9), cyclodecyl (C10); branched alkyls such as iso-propyl (C3), iso-butyl (C4), sec-butyl (C4), tert-butyl (C4), iso-pentyl (C5) and neo-pentyl (C5).

C2-C10 alkenyls include ethenyl (vinyl, —CH═CH2), 1-propenyl (—CH═CH—CH₃), 2-propenyl (allyl, —CH₂—CH═CH₂), isopropenyl (1-methylvinyl, —C(CH₃)═CH₂), butenyl (C4), pentenyl (C5), hexenyl (C6) and so forth; cyclic alkenyls include cyclopropenyl, cyclobutenyl (C4), cyclopentenyl (C5), cyclohexenyl (C6), cycloheptenyl (C7), cyclooctenyl (C8), cyclononenyl (C9), cyclodecenyl (C10).

According to an embodiment of the present invention, in the compound comprising a structure of formula (I), n is an integer of 1 to 4 and m=n.

According to another embodiment n is an integer from 1 to 4, m=n, L is (II), all n residues are selected from R1-R4, and, each of the n linkers comprises at least one S-, O-, N- or P-donor group.

Preferably, M is Ru^{II}, n=4, R1 to R4 are all identical and each is a linker comprising one S-, O-, N-, or P-donor group.

More specifically, the compound of formula (I) is [Ru₄(A)₄(B)₄(C)₄], with A, L, B, and C being defined as above.

According to a preferred embodiment of the present invention, the compound of the present invention is [Ru₄(A)₄(TPP)(B)₄(C)₄], wherein TPP is 5,10,15,20-tetra(4-pyridyl)porphyrin and A, B, C are defined as above. More preferably, the compound of the invention is [Ru₄(η⁶-arene)₄(TPP) Cl₈]. For example, (η⁶-arenes are selected from benzene (C₆H₆), methyl-benzene (C₆H₅CH₃), 1-isopropyl-4-methylbenzene (p-PrⁱC₆H₄Me), hexamethylbenzene (C₆Me₆) and diethyl terephtalate.

The compounds of the present invention may be used as pharmaceutically active principles. For example, they are useful as medicaments. The compounds of the present invention are preferably used as medicaments against cancer. They can also be used in the treatment of inflammatory and/or immune disorders, for example by targeting cells of the immune system, such as macrophages, neutrophils and/or lymphocytes.

Accordingly, the present invention relates to the use of the organometallic compounds comprising a structure of formula (I) in therapeutic cancer treatment. The invention also relates to the use of these compounds in the treatment of inflammatory and/or immune disorders.

The invention provides compounds comprising a structure of formula (I), or prodrugs or solvates thereof (“active compounds”), for use in a method of treatment of the human or animal body. A method of treatment may comprise administering to such an individual a therapeutically-effective amount of the compound of the present invention, preferably in the form of a pharmaceutical composition. The term treatment as used herein in the context of treating a condition, pertains generally to treatment and therapy, whether of a human or animal (e.g. in veterinary applications), in which some therapeutic effect is achieved, for example, the inhibition of the progress of the condition, and includes a reduction in the rate of the progress, a halt in the rate of the progress, amelioration of the condition, and cure of the condition. The condition usually is associated with suffering, from psychological and/or physical pain, with the individual being in need of a treatment. Treatment as a prophylactic measure (i.e. prophylaxis) is also included.

The compound of the invention or pharmaceutical composition comprising the active compound may be administered to an individual by any convenient route of administration, whether systemically/peripherically or at the site of desired action, including but not limited to, oral (e.g. by ingestion), topical (including e.g. transdermal, intranasal, ocular, buccal and sublingual), pulmonary (e.g. by inhalation or insufflation therapy using an aerosol, e.g. through mouth or nose), rectal, vaginal, parenteral, for example by injection, including subcutaneous, intradermal, intramuscular, intravenous, intraarterial, intracardiac, intrathecal, subcuticula, subcapsular, intraorbital, intraperitoneal, intratracheal, subarachnoid, and infrasternal, by implant of a depot (e.g. subcutaneously or intramuscularly).

The compound of the present invention is particularly useful in photo dynamic therapy (PDT).

With respect to the method of treatment of cancer according to the present invention, said method preferably comprises the further step of exposing said subject to light of a specific wavelength and/or intensity. The optimal wavelength will depend on various factors, such as the specific disease treated, the porphyrin or phtalocyanine core, and, in case of cancer treatment, the position of the cancer in the human body, for example. The skilled person will easily determine the optimal value. As a starting point, one can use wavelength of 400-800 nm, with a 200 mW halogen light source equivalent to a light exposure dose of 1.2 J/cm². The same applies for the treatment of inflammatory and/or immune disorders.

The compound of the present invention may be administered alone, but is preferably presented as a pharmaceutical composition (e.g. formulation) comprising at least one active compound together with at least one or more pharmaceutically carriers, buffers, as mentioned in WO 2006/018649, p. 16-20, “formulations”, which reference is expressly incorporated herein by reference.

Similar to the wavelength and light intensity, the optimal compound concentration will need to be determined, by the skilled person, in dependency of the disease treated, the treatment regime, and so forth. So far, plasma concentrations in the ranges of 0.5 to 1000 μM, preferably 1 to 500 μM and most preferably 5 to 100 μM are considered to be efficient.

The present invention also encompasses a method for the treatment of inflammatory and/or an immune disorders, the method comprising the steps of administering, to an individual in need thereof, an effective amount of one or more compounds according to the invention. According to an embodiment, the method further comprises the step of exposing a pertinent body part of the individual to irradiation. Irradiation may be as detailed above. A “body part”, for the purpose of the present invention, may be a tissue, organ, a group of cells, a limp, skin, and so forth. The body part may also be an extracellular fluid, such as serum, which has temporarily been removed from the body.

The present invention is described more concretely by way of the following examples, which, however, are not intended to be understood as any kind of restriction of the scope of the present invention.

### EXAMPLES

### Examples 1-10

### Synthesis and Characterisation of Compounds of the Present Invention

All organic solvents were degassed and saturated with nitrogen prior to use. 5,10,15,20-Tetra(4-pyridyl)porphyrin (TPP) was purchased from Fluka.

The following starting materials were prepared according to published methods, as indicated.
[Ru(η⁶-C₆H₆)(μ-Cl)Cl]₂: Zelonka, R. A.; Baird, M. C. Can. J. Chem., 1972, 50, 3063-3072.
[Ru(η⁶-C₆H₅CH₃)(μ-Cl)Cl]₂: Bennett, M. A.; Smith, A. K. J. Chem. Soc.; Dalton Trans., 1974, 233-241.
[Ru(η⁶-p-PrⁱC₆H₄Me)(μ-Cl)Cl]₂: Zelonka, R. A.; Baird, M. C. Can. J. Chem., 1972, 50, 3063-3072.
[Ru(η⁶-C₆Me₆)(μ-Cl)Cl]₂: Bennett, M. A.; Huang, T.-N.; Matheson, T. W.; Smith, A. K. Inorg. Synth.; John Wiley: New York, 1982, 21, p. 74-76.
[Ru(η⁶-1,4-C₆H₄(COOEt)₂)(μ-Cl)Cl]₂: Therrien, B.; Suss-Fink, G. Inorg. Chim. Acta, 2006, 359, 4350-4354.
[Os(η⁶-p-PrⁱC₆H₄Me)(μ-Cl)Cl]₂: Arthur, T.; Stephenson, T. A. J. Organomet. Chem., 1981, 208, 369-387.
[Rh(η⁵-Cp*)(μ-Cl)Cl]₂: White, C.; Oliver, A. J.; Maitlis, P. M. J. Chem. Soc.; Dalton Trans., 1973, 1901-1907.
[Ir(η⁵-Cp*)(μ-Cl)Cl]₂: White, C.; Oliver, A. J.; Maitlis, P. M. J. Chem. Soc.; Dalton Trans., 1973, 1901-1907.

NMR spectra were recorded on a Varian 200 MHz spectrometer. IR spectra were recorded on a Perkin-Elmer 1720×FT-IR spectrometer (4000-400 cm⁻¹). Microanalyses were performed by the Laboratory of Pharmaceutical Chemistry, University of Geneva (Switzerland). Electro-spray mass spectra were obtained in positive-ion mode with an LCQ Finnigan mass spectrometer.

### Example 1

### [Ru₄(η⁶-C₆H₆)₄(TPP)Cl₈] (1)

A mixture of [Ru(η⁶-C₆H₆)(μ-Cl)Cl]₂ (100 mg, 0.2 mmol) and 5,10,15,20-(pyrid-4-yl) porphyrin (TPP) (62 mg, 0.1 mmol) was refluxed in dry methanol (20 ml) for 4 h whereby the brownish purple-colored product was separated out. The compound was filtered and washed with diethylether and dried under vacuum. (Yield 90 mg, 56%). ¹H NMR (DMSO-d₆, 200 MHz): δ (ppm)=9.07 (d, 8H, ³J_{H-H}=5.88 Hz, H_{α}, pyridine), 8.92 (s, 8H, CH, pyrrole), 8.29 (d, 8H, H_{β}, pyridine), 6.52 (s, 24H, C₆H₆), −3.05 (s, 2H, NH). Elemental analysis (%) calc. for C₆₄H₅₀N₈Cl₈Ru₄: C, 47.48; H, 3.11; N, 6.92; found: C, 47.03, H, 3.17, N, 6.53.

### Example 2

### [Ru₄(η⁶-C₆H₅CH₃)₄(TPP)Cl₈] (2)

It was prepared in the same procedure as described above for 1 using [Ru(η⁶-C₆H₅CH₃)(μ-Cl)Cl]₂ (100 mg, 0.19 mmol) and TPP (59 mg, 0.095 mmol). (Yield 110 mg, 70%). ¹H NMR (DMSO-d₆, 200 MHz,): δ (ppm)=9.09 (d, 8H, ³J_{H-H}=4.76 Hz, H_{α}, pyridine), 8.93 (s, 8H, CH, pyrrole), 8.31 (d, 8H, H_{β}, pyridine), 6.03-5.97 (m, 12H, toluene), 5.72 (d, 8H, ³J_{H-H}=5.52 Hz, toluene), 2.52 (s, 12H, CH₃, toluene), −3.04 (s, 2H, NH). Elemental analysis (%) calc. for C₆₈H₅₈N₈Cl_{B}Ru₄: C, 48.75; H, 3.49; N, 6.69; found: C, 48.80, H, 3.70, N, 6.83.

### Example 3

### [Ru₄(η⁶-p-PrⁱC₆H₄Me)₄(TPP)Cl₈] (3)

It was prepared in the same procedure as described above for 1 using [Ru(η⁶-p-PrⁱC₆H₄Me)(μ-Cl)Cl]₂ (100 mg, 0.163 mmol) and TPP (50.5 mg, 0.082 mmol). (Yield 105 mg, 70%). ¹H NMR (DMSO-d₆, 200 MHz): δ (ppm)=9.07 (d, 8H, ³J_{H-H}=5.88 Hz, H_{α}, pyridine), 8.91 (s, 8H, CH, pyrrole), 8.29 (d, 8H, H_{β}, pyridine), 5.83 (d, 8H, ³J_{H-H}=6.6 Hz, CH, cymene), 5.78 (d, 8H, CH, cymene), 2.83 (sep, 4H, CH, cymene), 2.08 (s, 12H, CH₃, cymene), 1.21 (d, 24H, CH₃, cymene), −3.07 (s, 2H, NH). Elemental analysis (%) calc. for C₈₀H₈₂N₈Cl₈Ru₄: C, 52.12, H, 4.48, N, 6.08; found: C, 52.16; H, 4.51; N, 5.96.

### Example 4

### [Ru₄(η⁶-C₆Me₆)₄(TPP)Cl₈] (4)

It was prepared in the same procedure as described above for 1 using [Ru(η⁶-C₆Me₆)(μ-Cl)Cl]₂ (100 mg, 0.15 mmol) and TPP (46.3 mg, 0.075 mmol). (Yield 115 mg, 79%). ¹H NMR (CD₂Cl₂, 200 MHz): δ (ppm)=9.25 (d, 8H, ³J_{H-H}=6.62 Hz, H_{β}, pyridine), 8.89 (s, 8H, CH, pyrrole), 8.21 (d, 8H, H_{β}, pyridine), 2.14 (s, 72H, C₆Me₆), −3.00 (s, 2H, NH). Elemental analysis (%) calc. for C₈₈H₉₈N₈Cl₈Ru₄: C, 54.04; H, 5.05; N, 5.73; found: C, 54.67, 4.90, N, 5.16.

### Example 5

### [Ru₄(η⁶-1,4-C₆H₄(COOEt)₂)₄(TPP)Cl₈] (5)

It was prepared in the same procedure as described above for 1 using [Ru(η⁶-1,4-C₆H₄(COOEt)₂)(μ-Cl)Cl]₂ (118 mg, 0.15 mmol) and TPP (46 mg, 0.075 mmol). (Yield 120 mg, 73%). NMR (CD₂Cl₂, 200 MHz): δ (ppm)=9.37 (d, 8H, ³J_{H-H}=5.44 Hz, H_{α}, pyridine), 8.95 (s, 8H, CH, pyrrole), 8.19 (d, 8H, H_{D}, pyridine), 5.73 (s, 16H, C₆H₄), 4.28-4.17 (q, 8H, ³J_{H-H}=6.98 Hz, CH₂), 1.33-1.26 (t, 12H, ³J_{H-H}=6.98 Hz, CH₃), −3.21 (s, 2H, NH). Elemental analysis (%) calc. for C₈₈H₈₂N₈Cl₈O₁₆Ru₄: C, 48.14, H, 3.76, N, 5.10; found: C, 48.35, H, 3.84, 4.95.

### Example 6

### [OS₄(η⁶-p-PrⁱC₆H₄Me)₄(TPP)Cl₈] (6)

It was prepared in the same procedure as described above for 1 using [Os(η⁶-p-PrⁱC₆H₄Me)(μ-Cl)Cl]₂ (150 mg, 0.214 mmol) and TPP (66 mg, 0.11 mmol). (Yield 110 mg, 47%). ¹H NMR (DMSO-d₆, 200 MHz): δ (ppm)=9.07 (d, 8H, ³J_{H-H}=5.88 Hz, H_{α}, pyridine), 8.92 (s, 8H, CH, pyrrole), 8.29 (d, 8H, H_{β}, pyridine), 6.09 (d, 8H, ³J_{H-H}=5.88 Hz, CH, cymene), 6.00 (d, 8H, CH, cymene), 2.77 (sep, 4H, ³J_{H-H}=6.98 Hz, CH, cymene), 2.13 (s, 12H, CH₃, cymene), 1.21 (d, 24H, CH₃, cymene), −3.06 (s, 2H, NH). Elemental analysis (%) calc. for C₈₀H₈₂N₈Cl₈Os₄: C, 43.68; H, 3.76; N, 5.09; found: C, 43.80, H, 3.98, N, 5.15.

### Example 7

### [Rh₄(η⁵-Cp*)₄(TPP)Cl₈] (7) (Cp*=pentamethylcyclopentadienyl)

It was prepared in the same procedure as described above for 1 using [Rh(η⁵-Cp*)(μ-Cl)Cl]₂ (100 mg, 0.16 mmol) and TPP (50 mg, 0.08 mmol). (Yield 110 mg, 73%). ¹H NMR (200 MHz, CD₂Cl₂): δ (ppm)=9.42 (d, 8H, ³J_{H-H}=6.24 Hz, H_{α}, pyridine), 8.87 (s, 8H, CH, pyrrole), 8.28 (d, 8H, Ho, pyridine), 1.53 (s, 60H, C₅Me₅), −3.01 (s, 2H, NH). Elemental analysis (%) calc. for C₈₀H₈₆N₈Cl₈Rh₄: C, 51.80; H, 4.67; N, 6.04; found: C, 51.61, H, 5.00, N, 6.24.

### Example 8

### [Ir₄(η⁵-Cp*)₄(TPP)Cl₈] (8)

It was prepared in the same procedure as described above for 1 using [Ir(η⁵-Cp*)(μ-Cl)Cl]₂ (100 mg, 0.126 mmol) and TPP (39 mg, 0.06 mmol). (Yield 115 mg, 83%). ¹H NMR (DMSO-d₆, 200 MHz,): δ (ppm)=9.07 (d, 8H, ³J_{H-H}=5.50 Hz, H_{α}, pyridine), 8.92 (s, 8H, CH, pyrrole), 8.29 (d, 8H, H_{β}, pyridine), 1.63 (s, 60H C₅Me₅), −3.06 (s, 2H, NH). Elemental analysis (%) calc. for C₈₀H₈₆N₈Cl₈Rh₄: C, 43.43; H, 3.92; N, 5.06; found: C, 43.64, H, 3.82, N, 5.65.

### Example 9

### UV-Visible Absorption Spectra

Table 1 shows UV-visible absorption maxima and molar extinction coefficients [λ(ε×10⁻³ M⁻¹ cm⁻¹)], determined in CH₂Cl₂ and fluorescence quantum yields (φ_{f}) of photosensitizers in MeOH at 648 nm after 410 nm excitation. The UV-visible absorption spectra were recorded on an Uvikon 930 spectrophotometer and fluorescence spectra on a Perkin-Elmer LS50 spectrofluorometer.

TABLE 1
UV-visible absorption data of selected compounds in dichloromethane at 298 K

| | Soret Band | Q Band IV | Q Band III | Q Band II | Q Band I | φ_{f} (%) |
|---|---|---|---|---|---|---|
| TPP | 417 (212.9) | 512 (24.2) | 545 (9.4) | 587 (9.8) | 642 (5.7) | 10.4 |
| 1 | 427 (198.5) | 515 (21.7) | 550 (8.6) | 590 (9.5) | 645 (7.1) | 6.1 |
| 2 | 423 (156.5) | 516 (13.6) | 550 (7.7) | 590 (6.7) | 645 (4.4) | 7.4 |
| 3 | 422 (188.2) | 515 (19.8) | 550 (10.1) | 590 (6.9) | 645 (4.8) | 7.9 |
| 4 | 422 (200.0) | 515 (18.7) | 550 (8.0) | 589 (6.2) | 645 (2.7) | 7.3 |
| 5 | 423 (208.8) | 517 (30.3) | 551 (18.6) | 590 (15.8) | 647 (10.3) | 7.2 |
| 6 | 418 (183.4) | 514 (25.5) | 548 (11.1) | 588 (9.7) | 643 (6.6) | 7.0 |
| 7 | 423 (216.2) | 514 (26.7) | 549 (12.1) | 589 (10.6) | 648 (5.7) | 7.9 |
| 8 | 418 (203.9) | 513 (15.8) | 547 (11.7) | 588 (8.3) | 644 (7.3) | 4.5 |

### Example 10

### X-Ray Crystallography

Single-crystals of (4) and (7) were mounted on a Stoe Image Plate Diffraction system equipped with a (1) circle goniometer, using Mo—Kα graphite monochromated radiation (λ=0.71073 Å) with φ range 0-200°, Dₘₐₓ−Dₘᵢₙ=12.45-0.81 Å, increment of 0.8 and 1.0°, respectively. The structures were solved by direct methods using the program SHELXS-97 (Sheldrick, G. M. Acta Cryst. 1990, A46, 467-473.) The refinement and all further calculations were carried out using SHELXL-97 (G. M. Sheldrick, SHELXL-97, University of Gottingen, Göttingen, Germany (1999)). In all cases, the H-atoms were included in calculated positions and treated as riding atoms using the SHELXL default parameters. Examination of the structures with PLATON (Spek, A. L. J. Appl. Cryst. 2003, 36, 7-13) reveals voids between the arene ruthenium porphyrin molecules. Indeed, voids corresponding to solvent molecules were found. Therefore, new data sets corresponding to omission of the missing solvents were generated with the SQUEEZE algorithm (van der Sluis, P.; Spek, A. L. Acta Cyst. 1990, A46, 194-201) and the two structures were refined to convergence. However, in both cases the non-H atoms were refined anisotropically, using weighted full-matrix least-square on F². Crystallographic details are summarized in Table 2.

TABLE 2
Crystallographic and selected experimental data for (4) and (7).

| | 4 | 7 |
|---|---|---|
| Chemical formula | C₈₈H₉₈Cl₈N₈Ru₄ | C₈₀H₈₆Cl₈N₈Rh₄ |
| Formula weight | 1955.62 | 1854.81 |
| Crystal system | Monoclinic | Triclinic |
| Space group | P 2₁/n | P −1 |
| Crystal color and shape | purple rod | purple block |
| Crystal size | 0.18 × 0.10 × 0.10 | 0.15 × 0.14 × 0.12 |
| a (Å) | 14.890(3) | 12.5575(13) |
| b (Å) | 16.0272(12) | 16.237(2) |
| c (Å) | 23.640(3) | 16.9186(19) |
| α (°) | | 64.686(13) |
| β (°) | 92.25(2) | 73.953(12) |
| γ (°) | | 71.819(13) |
| V (Å³) | 5637.3(15) | 2921.3(6) |
| Z | 2 | 1 |
| T (K) | 173(2) | 173(2) |
| D_{c} (g · cm⁻³) | 1.152 | 1.054 |
| μ (mm⁻¹) | 0.753 | 0.771 |
| Scan range (°) | 4.16 < 2θ < 51.86 | 3.96 < 2θ < 51.86 |
| Unique reflections | 10008 | 10590 |
| Reflections used [I > 2σ(I)] | 3384 | 3257 |
| Rᵢₙₜ | 0.1747 | 0.1659 |
| Final R indices [I > 2σ(I)]* | 0.0896, wR₂ 0.2111 | 0.0835, wR₂ 0.1955 |
| R indices (all data) | 0.1778, wR₂ 0.2353 | 0.1788, wR₂ 0.2251 |
| Goodness-of-fit | 0.736 | 0.689 |
| Max, Min Δρ/e (Å⁻³) | 1.458, −1.014 | 1.027, −1.342 |

*Structures were refined on F₀²: wR₂ = [Σ[w(F₀² − F_{c}²)²]/Σw(F₀²)²]^{1/2}, where w⁻¹ = [Σ(F₀²) + (aP)² + bP] and P = [max(F₀², 0) + 2F_{c}²]/3

Selected bond lengths (Å) and angles)(°) of compound 4: Ru(1)-C1(1) 2.416 (4), Ru(1)-C1(2) 2.404(3), Ru(1)-N(1) 2.162 (7), Ru(2)-C1(3) 2.450(4), Ru(2)-Cl(4) 2.395 (3), Ru(2)-N(2) 2.100(10); C1(1)-Ru(1)-C1(2) 87.67 (13), N(1)-Ru(1)-C1(1) 87.2 (3), N(1)-Ru(1)-C1(2) 84.3(3), Cl(3)-Ru(2)-Cl(4) 88.47 (14), N(2)-Ru(2)-Cl(3) 82.2 (3), N(2)-Ru(2)-C1(4) 85.8(2).

Selected bond lengths (Å) and angles)(°) of compound 7: Rh(1)-Cl(1) 2.405 (3), Rh(1)-C1(2) 2.425 (4), Rh(1)-N(1) 2.154(9), Rh(2)-Cl(3) 2.428 (4), Rh(2)-Cl(4) 2.431(3), Rh(2)-N(2) 2.103 (10); C1(1)-Rh(1)-C1(2) 92.30 (13), N(1)-Rh(1)-C1(1) 88.0 (3), N(1)-Rh(1)-Cl(2) 87.0(3), C1(3)-Rh(2)-C1(4) 91.72 (11), N(2)-Rh(2)-C1(3) 86.7 (3), N(2)-Rh(2)-C1(4) 89.2(3).

### Examples 11

### Toxicity of the Present Compounds on Melanoma Cancer Cell Lines

Human ME275 and ME300 melanoma cell lines were from Dr D. Rimoldi, Ludwig Institute of Cancer Research, Lausanne. All other cell culture reagents were obtained from Gibco-BRL, Basel Switzerland. The cells were routinely grown in RPMI 1640 medium containing 10% foetal calf serum (FCS) and antibiotics. The organometallic complexes were dissolved in DMSO as 10 mM stock solution and then diluted in complete medium to the required concentration. DMSO at comparable concentrations did not show any effects on cell cytotoxicity (results not shown). To determine the cytotoxicity of the ruthenium compounds in the dark, cells were grown in 48-well cell culture plastic plates (Corning, N.Y.) until 75% confluent. The culture medium was replaced with fresh medium containing complexes 1-7 for concentrations varying from 0 to 100 μM and cells were exposed to the complexes for 24 h. Thereafter, the medium was replaced by fresh medium and cell survival was measured using the MTT test as previously described (Berger, Y. et al. J. Med. Chem. 2000, 43, 4738-46). Briefly, 3-(4,5-dimethyl-2-thiazoyl)-2,5-diphenyltetrazolitun bromide (MTT, Merck) was added at 250 μg/mL and incubation was continued for 2 h, then the cell culture supernatants were removed, the cell layer were dissolved in iPrOH/0.04N HCl, absorbance at 540 nm were measured in a 96-well multiwell-plate reader (iEMS Reader MF, Labsystems, Bioconcept, Switzerland) and compared to the values of control cells incubated without complexes. Experiments were conducted in triplicate wells and repeated at least twice.

Results of non-light irradiated Me300 melanoma cells are shown in FIG. 4. It can be seen that most of the compounds strongly reduce cell viability, even in absence of irradiation, and that cytotoxicity increases with concentration.

Analoguous experiments conducted with human ME275 melanoma cells yielded similar results.

### Example 12

### Phototoxicity

For determining phototoxicity, cells were grown in multiwell cell culture plastic plates (Corning, N.Y.) until 75% confluent. The culture media were replaced with fresh medium containing complexes 1-7 at 10 μM concentration (minimal dark cytotoxicity) and the cells were exposed to the complexes for 24 h. Thereafter, the media were replaced with RPMI without phenol red containing 5% FCS and cells were irradiated at 652 nm using a diode laser (Applied Optronics, South Plainfield, N.J.) coupled to a frontal diffuser (Medlight S A, Ecublens, Switzerland), at an irradiance of 20 mW/cm² and light doses ranging between 5 and 30 J/cm². Experiments were conducted in triplicate. Analysis of cell phototoxicity using the MTT assay as described above was performed after a further incubation of 24 h after irradiation and compared to the values of control cells without laser irradiation.

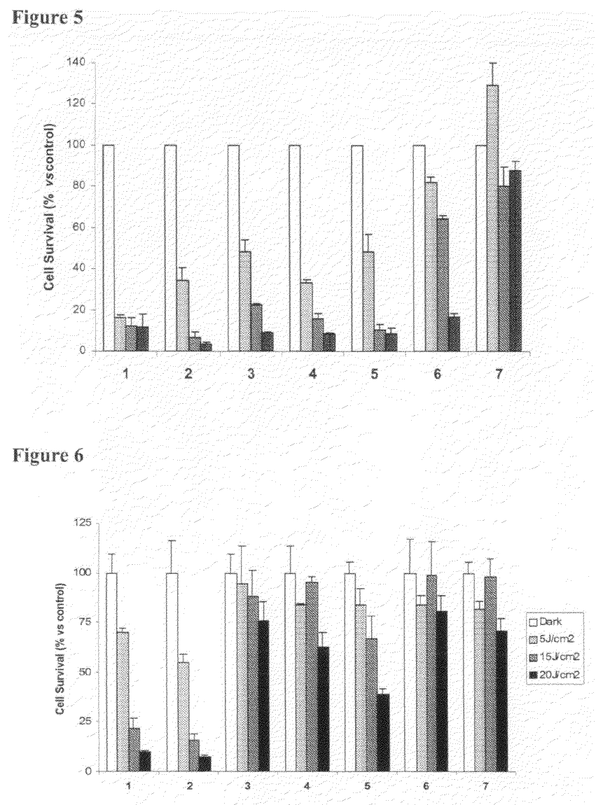

The results are shown in FIG. 5. Light intensities: 0 J/cm² (white bars), 5 J/cm² (light grey bars), 15 J/cm² (dark grey bars) and 30 J/cm² (black bars), respectively. For all compounds, except for compound 7, in which Rh served as metal nucleus, phototoxicity increased with increasing doses light but high phototoxicity was generally obtained already with low irradiation intensity.

Similarly significant results were obtained when testing compounds 1-7 on the human ME 275 melanoma cell line.

### Example 13

### Anti-inflammatory Properties of the Compounds of the Invention

### Methods: Phototoxicity Experiments

RAW cells were grown in 96-well cell culture plates (Corning, N.Y.) until 75% confluent in DMEM medium containing 10% FCS. The culture media were replaced with fresh medium containing the Ru, Os or Rh-porphyrin analogs at 10 μM (minimal dark cytotoxicity) and the cells were exposed overnight to the compounds. Then, the media were replaced with DMEM without phenol red containing 10% FCS and cells were irradiated at 652 nm using a diode laser (Applied Optronics, South Plainfield, N.J.) coupled to a frontal diffuser (Medlight SA, Ecublens, Switzerland), at an irradiance of 20 mW/cm². Experiments were performed in triplicate wells. Cell viability was evaluated 24 h later using the MTT test. Briefly, 3-(4,5-dimethyl-2-thiazoyl)-2,5-diphenyltetrazolium bromide (MTT, Merck) was added at 250 μg/mL and incubation was continued for 2 h, then the cell culture supernatants were removed. The cell layers were dissolved in iPrOH/0.04N HCl and the absorbance measured at 540 nm in a multiwell-plate reader (iEMS Reader MF, Labsystems, Bioconcept, Switzerland) and compared to the values of control cells incubated with the nanoparticles but without laser irradiation.

### Results

Photosensitivity induced by the different Ru, Os and Rh-porphyrin analogs (FIG. 2) was evaluated after 24 h of incubation (FIG. 6). None of the analogs was cytotoxic in the absence of exposure to light. Compounds 1 and 2 analogs were the best photosensitizers amongst the Ru-porphyrin compounds.

1-14. (canceled)

15. An organometallic compound comprising a structure of formula (I):
[MₙMc(A)ₙ(L)(B)ₚ(C)_{q}] (I)
which is charged or neutral and which may be present in the form of a salt and/or an optically resolved enantiomer, and,
wherein:
M is a transition metal selected from Ru, Rh, Os, Ir, and Fe, preferably Ru;
n is an integer of 1 to 12 if L corresponds to formula (II) below and an integer of 1 to 16 if L corresponds to formula (III) below and designs the number of metal atoms M present in compound (I);
Mc is an optional metal in the centre of L, which may be selected from the same metals as M, and in addition from the metals Zn, Ni, Co, Cu, Ag, Au, Cd, Hg, Pd, Pt.
A and L are ligands of M;
B and C are, independently of each other, optional ligands of M, with p and q, independently of each other, being 0 or an integer of 1 to n.
wherein, if n>1, all n ligands A and all n metals M in the compound of formula (I) can be the same or different; and, accordingly, wherein, if p and/or q>1, all p ligands B and/or all q ligands B, respectively, in the compound of formula (I) can be the same or different;
A is an η⁶-arene or a η⁵-cyclopentadienyl, or a η⁵-cyclopentadienyl derivative;
B and C are, independently of each other, freely selectable ligands capable of binding to M,
wherein:
B and C may be covalently connected with each other so as to form a single, bidentate chelating ligand (B-C), or, alternatively,
any one or both of B and C, may be covalently bound to A, so as to form a multidentate chelating ligand (A-C), (A-B) or (A-B-C);
L is a porphyrin derivative of formula (II) or a phtalocyanine derivative of formula (III) below: 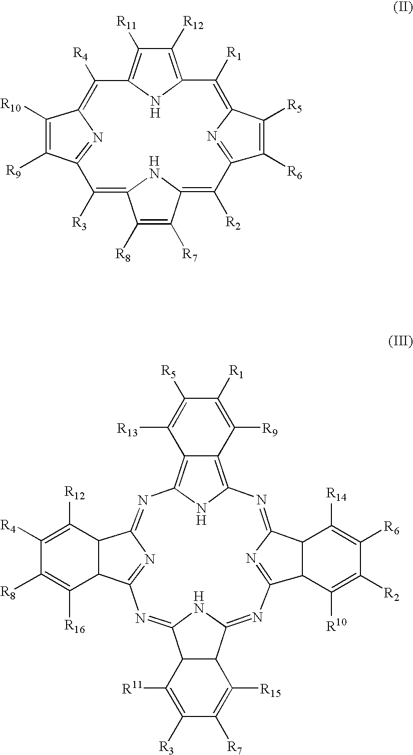
wherein:
m residues of R₁-R₁₂ if L=(II) or of R₁-R₁₆ if L=(III) are linkers comprising, all m residues together, at least n donor groups for M, wherein each of said at least n donor groups is selected independently from S-, O-, N- and/or P-donor groups, and,
m is an integer of 1 to 12 if L=(II) and an integer of 1 to 16 if L=(III),
wherein:
two or more (r) of said m residues of R₁-R₁₂ if L=(II) or of R₁-R₁₆ if L=(III) may, independently of the remaining (m-r) residues, be covalently connected with each other to form a system comprising at least one ring fused to the porphyrin structure of formula (II) or to the phtalocyanine structure of formula (III), respectively;
and, wherein:
(12-m) residues of R₁-R₁₂ if L=(II) or (16-m) of R₁-R₁₆ if L=(III), respectively, are selected, independently of each other, from H, C1-C20 alkyls, C2-20 alkenyls, C2-C20 alkynyl, C6-C22 aryls, said alkyls, alkenyls, alkynyls and aryls being optionally substituted and optionally comprising one or more heteroatoms, said optionally substituted alkyls, alkenyls, alkynyls being linear or cyclic.

16. The organometallic compound of claim 15, wherein A is a compound of formula (IV) or (V): 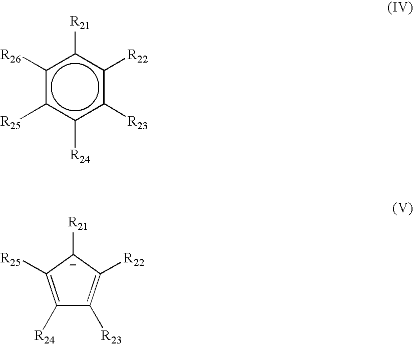
wherein, if A is (IV), any one of R₂₁-R₂₆, or, if A is (V), respectively, any one of R₂₁-R₂₅, is independently selected from H, C1-C20 alkyl, C2-20 alkenyl, C2-C20 alkynyl, C6-C22 aryl, said alkyl, alkenyl, alkynyl being linear or cyclic, and said alkyl, alkenyl, alkynyl and aryls being optionally substituted and optionally comprising one or more heteroatoms,
wherein any of the residues R₂₁-R₂₆ or R₂₁-R₂₅, respectively may be covalently connected with one or more other residues of R₂₁-R₂₆ or R₂₁-R₂₅, respectively, to form one or several rings fused to the ring of formula (IV) or (V), respectively.

17. The organometallic compound of claim 15, wherein:
n is an integer from 1 to 4,
m=n,
L is (II),
all n residues are selected from R1-R4, and,
each of the n linkers comprises at least one S-, O-, N- or P-donor group.

18. The organometallic compound of claim 15, wherein each of said linkers comprises, independently from each other, at least one aromatic N-ring or at least one amine functioning as said N-donor group.

19. The organometallic compound of claim 15, wherein A and B are, independently of each other, selected from H, F, Cl, Br, I, OH, OMe, OEt, OBu, OH₂, CH₃CN, (CH₃)₂CO, DMSO, THF, CO, CF₃SO₃, C₂O₄, PMe₃, PEt₃, PBu₃, PPh₃, PCy₃, HPPh₂, HPCy₂, acetate, acetylacetonate, pyridine, pyrimidine, pyrazine, imidazole, pyridazine, phthlalazine, quinoxaline, 1,5-naphthyridine, 1,8-naphthyridine, 2,2′-bipyrimidine, phenazine, azobenzene, 2,2′-azopyridine, 2,3-di-(2′-pyridino)quinoxaline, diphenylphosphineethane, pyrazinecarboxylic acid, pyrazinedicarboxylic acid, DABCO, ethylenediamine, 1,3,5-triaza-7-phosphatricyclo [3.3.1.1^{3,7}]decane.

20. The organometallic compound of claim 15, wherein M is R^{II}, n=m=4, R1 to R4 are all identical and each is a linker comprising one S-, O-, N- or P-donor group.

21. The organometallic compound of claim 15, which is [Ru₄A₄(L)B₄C₄], with A, B, and C being defined as above.

22. The organometallic compound of claim 15, which is [Ru₄(η⁶-arene)₄(TPP)Cl₈], wherein TPP is 5,10,15,20-tetra(4-pyridyl)porphyrin.

23. The organometallic compound of claim 15 for use as a pharmaceutically active principle, in particular as a medicament.

24. A method for the treatment of cancer, the method comprising the steps of administering, to an individual in need thereof, an effective amount of one or more organometallic compounds comprising a structure of formula (I):
[MₙMc(A)(L)(B)ₚ(C)_{q}] (I)
which is charged or neutral and which may be present in the form of a salt and/or an optically resolved enantiomer, and,
wherein:
M is a transition metal selected from Ru, Rh, Os, Ir, and Fe, preferably Ru;
n is an integer of 1 to 12 if L corresponds to formula (II) below and an integer of 1 to 16 if L corresponds to formula (III) below and designs the number of metal atoms M present in compound (I);
Mc is an optional metal in the centre of L, which may be selected from the same metals as M, and in addition from the metals Zn, Ni, Co, Cu, Ag, Au, Cd, Hg, Pd, Pt.
A and L are ligands of M;
B and C are, independently of each other, optional ligands of M, with p and q, independently of each other, being 0 or an integer of 1 to n.
wherein, if n>1, all n ligands A and all n metals M in the compound of formula (I) can be the same or different; and, accordingly, wherein, if p and/or q>1, all p ligands B and/or all q ligands B, respectively, in the compound of formula (I) can be the same or different;
A is an η⁶-arene or a η⁵-cyclopentadienyl, or a η⁵-cyclopentadienyl derivative;
B and C are, independently of each other, freely selectable ligands capable of binding to M,
wherein:
B and C may be covalently connected with each other so as to form a single, bidentate chelating ligand (B-C), or, alternatively,
any one or both of B and C, may be covalently bound to A, so as to form a multidentate chelating ligand (A-C), (A-B) or (A-B-C);
L is a porphyrin derivative of formula (II) or a phtalocyanine derivative of formula (III) below: 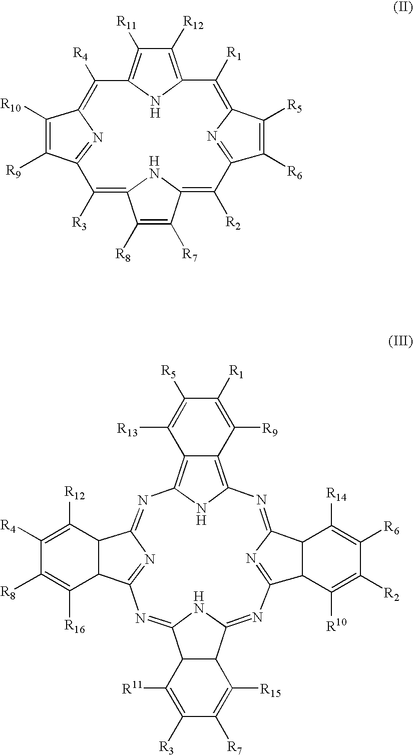
wherein:
m residues of R₁-R₁₂ if L=(II) or of R₁-R₁₆ if L=(III) are linkers comprising, all m residues together, at least n donor groups for M, wherein each of said at least n donor groups is selected independently from S-, O-, N- and/or P-donor groups, and,
m is an integer of 1 to 12 if L=(II) and an integer of 1 to 16 if L=(III),
wherein:
two or more (r) of said m residues of R₁-R₁₂ if L=(II) or of R₁-R₁₆-if L=(III) may, independently of the remaining (m-r) residues, be covalently connected with each other to form a system comprising at least one ring fused to the porphyrin structure of formula (II) or to the phtalocyanine structure of formula (III), respectively;
and, wherein:
(12-m) residues of R₁-R₁₂ if L=(II) or (16-m) of R₁-R₁₆ if L=(III), respectively, are selected, independently of each other, from H, C1-C20 alkyls, C2-20 alkenyls, C2-C20 alkynyl, C6-C22 aryls, said alkyls, alkenyls, alkynyls and aryls being optionally substituted and optionally comprising one or more heteroatoms, said optionally substituted alkyls, alkenyls, alkynyls being linear or cyclic.

25. The method according to claim 24, the method comprising the steps of administering, to an individual in need thereof, an effective amount of one or more organometallic compounds of formula (I), wherein A is a compound of formula (IV) or (V): 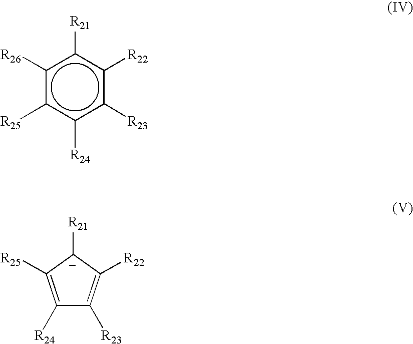
wherein, if A is (IV), any one of R₂₁-R₂₆, or, if A is (V), respectively, any one of R₂₁-R₂₅, is independently selected from H, C1-C20 alkyl, C2-20 alkenyl, C2-C20 alkynyl, C6-C22 aryl, said alkyl, alkenyl, alkynyl being linear or cyclic, and said alkyl, alkenyl, alkynyl and aryls being optionally substituted and optionally comprising one or more heteroatoms,
wherein any of the residues R₂₁-R₂₆ or R₂₁-R₂₅, respectively may be covalently connected with one or more other residues of R₂₁-R₂₆ or R₂₁-R₂₅, respectively, to form one or several rings fused to the ring of formula (IV) or (V), respectively.

26. The method according to claim 24, the method comprising the steps of administering, to an individual in need thereof, an effective amount of one or more organometallic compounds of formula (I), wherein:
n is an integer from 1 to 4,
m=n,
L is (II),
all n residues are selected from R1-R4, and,
each of the n linkers comprises at least one S-, O-, N- or P-donor group.

27. The method according to claim 24, the method comprising the steps of administering, to an individual in need thereof, an effective amount of one or more organometallic compounds of formula (I), wherein each of said linkers comprises, independently from each other, at least one aromatic N-ring or at least one amine functioning as said N-donor group.

28. The method according to claim 24, the method comprising the steps of administering, to an individual in need thereof, an effective amount of one or more organometallic compounds of formula (I), wherein A and B are, independently of each other, selected from H, F, Cl, Br, I, OH, OMe, OEt, OBu, OH₂, CH₃CN, (CH₃)₂CO, DMSO, THF, CO, CF₃SO₃, C₂O₄, PMe₃, PEt₃, PBu₃, PPh₃, PCy₃, HPPh₂, HPCy₂, acetate, acetylacetonate, pyridine, pyrimidine, pyrazine, imidazole, pyridazine, phthlalazine, quinoxaline, 1,5-naphthyridine, 1,8-naphthyridine, 2,2′-bipyrimidine, phenazine, azobenzene, 2,2′-azopyridine, 2,3-di-(2′-pyridino)quinoxaline, diphenylphosphineethane, pyrazinecarboxylic acid, pyrazinedicarboxylic acid, DABCO, ethylenediamine, 1,3,5-triaza-7-phosphatricyclo[3.3.1.1^{3,7}]decane.

29. The method according to claim 24, the method comprising the steps of administering, to an individual in need thereof, an effective amount of one or more organometallic compounds of formula (I), wherein M is R^{II}, n=m=4, R1 to R4 are all identical and each is a linker comprising one S-, O-, N- or P-donor group.

30. The method according to claim 24, the method comprising the steps of administering, to an individual in need thereof, an effective amount of one or more organometallic compounds of formula (I), which is [Ru₄A₄(L)B₄C₄], with A, B, and C being defined as above.

31. The method according to claim 24, the method comprising the steps of administering, to an individual in need thereof, an effective amount of one or more organometallic compounds of formula (I), which is [Ru₄ (η⁶-arene)₄ (TPP) Cl₈], wherein TPP is 5,10,15,20-tetra(4-pyridyl)porphyrin.

32. A method for the treatment of inflammatory and/or an immune disorder, the method comprising the steps of administering, to an individual in need thereof, an effective amount of one or more compounds organometallic compounds comprising a structure of formula (I):
[MₙMc(A)(L)(B)ₚ(C)_{q}] (I)
which is charged or neutral and which may be present in the form of a salt and/or an optically resolved enantiomer, and,
wherein:
M is a transition metal selected from Ru, Rh, Os, Ir, and Fe, preferably Ru;
n is an integer of 1 to 12 if L corresponds to formula (II) below and an integer of 1 to 16 if L corresponds to formula (III) below and designs the number of metal atoms M present in compound (I);
Mc is an optional metal in the centre of L, which may be selected from the same metals as M, and in addition from the metals Zn, Ni, Co, Cu, Ag, Au, Cd, Hg, Pd, Pt.
A and L are ligands of M;
B and C are, independently of each other, optional ligands of M, with p and q, independently of each other, being 0 or an integer of 1 to n.
wherein, if n>1, all n ligands A and all n metals M in the compound of formula (I) can be the same or different; and, accordingly, wherein, if p and/or q>1, all p ligands B and/or all q ligands B, respectively, in the compound of formula (I) can be the same or different;
A is an η⁶-arene or a η⁵-cyclopentadienyl, or a η⁵-cyclopentadienyl derivative;
B and C are, independently of each other, freely selectable ligands capable of binding to M,
wherein:
B and C may be covalently connected with each other so as to form a single, bidentate chelating ligand (B-C), or, alternatively,
any one or both of B and C, may be covalently bound to A, so as to form a multidentate chelating ligand (A-C), (A-B) or (A-B-C);
L is a porphyrin derivative of formula (II) or a phtalocyanine derivative of formula (III) below: 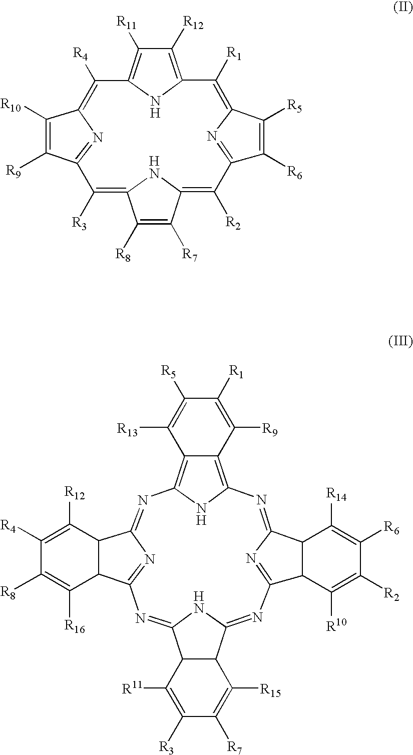
wherein:
m residues of R₁-R₁₂ if L=(II) or of R₁-R₁₆ if L=(III) are linkers comprising, all m residues together, at least n donor groups for M, wherein each of said at least n donor groups is selected independently from S-, O-, N- and/or P-donor groups, and,
m is an integer of 1 to 12 if L=(II) and an integer of 1 to 16 if L=(III),
wherein:
two or more (r) of said m residues of R₁-R₁₂ if L=(II) or of R₁-R₁₆ if L=(III) may, independently of the remaining (m-r) residues, be covalently connected with each other to form a system comprising at least one ring fused to the porphyrin structure of formula (II) or to the phtalocyanine structure of formula (III), respectively;
and, wherein:
(12-m) residues of R₁-R₁₂ if L=(II) or (16-m) of R₁-R₁₆ if L=(III), respectively, are selected, independently of each other, from H, C1-C20 alkyls, C2-20 alkenyls, C2-C20 alkynyl, C6-C22 aryls, said alkyls; alkenyls, alkynyls and aryls being optionally substituted and optionally comprising one or more heteroatoms, said optionally substituted alkyls, alkenyls, alkynyls being linear or cyclic.

33. The method according to claim 32, the method comprising the steps of administering, to an individual in need thereof, an effective amount of one or more compounds organometallic compounds comprising a structure of formula (I), wherein A is a compound of formula (IV) or (V): 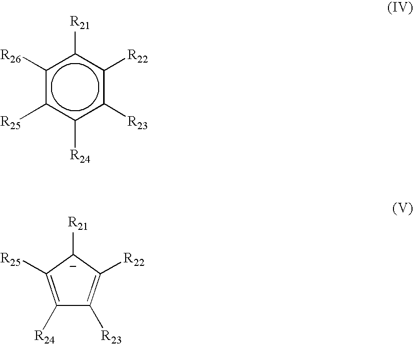
wherein, if A is (IV), any one of R₂₁-R₂₆, or, if A is (V), respectively, any one of R₂₁-R₂₅, is independently selected from H, C1-C20 alkyl, C2-20 alkenyl, C2-C20 alkynyl, C6-C22 aryl, said alkyl, alkenyl, alkynyl being linear or cyclic, and said alkyl, alkenyl, alkynyl and aryls being optionally substituted and optionally comprising one or more heteroatoms,
wherein any of the residues R₂₁-R₂₆ or R₂₁-R₂₅, respectively may be covalently connected with one or more other residues of R₂₁-R₂₆ or R₂₁-R₂₅, respectively, to form one or several rings fused to the ring of formula (IV) or (V), respectively.

34. The method according to claim 32, the method comprising the steps of administering, to an individual in need thereof, an effective amount of one or more compounds organometallic compounds comprising a structure of formula (I), wherein:
n is an integer from 1 to 4,
m=n,
L is (II),
all n residues are selected from R1-R4, and,
each of the n linkers comprises at least one S-, O-, N- or P-donor group.

35. The method according to claim 32, the method comprising the steps of administering, to an individual in need thereof, an effective amount of one or more compounds organometallic compounds comprising a structure of formula (I), wherein each of said linkers comprises, independently from each other, at least one aromatic N-ring or at least one amine functioning as said N-donor group.

36. The method according to claim 32, the method comprising the steps of administering, to an individual in need thereof, an effective amount of one or more compounds organometallic compounds comprising a structure of formula (I), wherein A and B are, independently of each other, selected from H, F, Cl, Br, I, OH, OMe, OEt, OBu, OH₂, CH₃CN, (CH₃)₂CO, DMSO, THF, CO, CF₃SO₃, C₂O₄, PMe₃, PEt₃, PBu₃, PPh₃, PCy₃, HPPh₂, HPCy₂, acetate, acetylacetonate, pyridine, pyrimidine, pyrazine, imidazole, pyridazine, phthlalazine, quinoxaline, 1,5-naphthyridine, 1,8-naphthyridine, 2,2′-bipyrimidine, phenazine, azobenzene, 2,2′-azopyridine, 2,3-di-(2′-pyridino)quinoxaline, diphenylphosphineethane, pyrazinecarboxylic acid, pyrazinedicarboxylic acid, DABCO, ethylenediamine, 1,3,5-triaza-7-phosphatricyclo[3.3.1.1^{3,7}]decane.

37. The method according to claim 32, the method comprising the steps of administering, to an individual in need thereof, an effective amount of one or more compounds organometallic compounds comprising a structure of formula (I), wherein M is R^{II}, n=m=4, R1 to R4 are all identical and each is a linker comprising one S-, O-, N- or P-donor group.

38. The method according to claim 32, the method comprising the steps of administering, to an individual in need thereof, an effective amount of one or more compounds organometallic compounds comprising a structure of formula (I), which is [Ru₄A₄(L)B₄C₄], with A, B, and C being defined as above.

39. The method according to claim 32, the method comprising the steps of administering, to an individual in need thereof, an effective amount of one or more compounds organometallic compounds comprising a structure of formula (I), which is [Ru₄ (η⁶-arene)₄ (TPP) Cl₈], wherein TPP is 5,10,15,20-tetra(4-pyridyl)porphyrin.

40. The method according to claim 24, comprising the step of exposing a pertinent body part of the individual to irradiation.

41. The method according to claim 32, comprising the step of exposing a pertinent body part of the individual to irradiation.
